(19) United States
(12) Patent Application Publication
(10) Pub. No.: US 2006/0063989 A1
(43) Pub. Date: Mar. 23, 2006

(54) Compact non-invasive analysis system

(76) Inventor: Josh N. Hogan, Los Altos, CA (US)

Correspondence Address: Josh Hogan, 620 Kingswood Way, Los Altos, CA, 94022, US

(21) Appl. No.: 10/949,917

(22) Filed: Sep. 25, 2004

Related U.S. Application Data
(60) Provisional application No. 60/602,913, filed on Aug. 19, 2004.

(51) Int. Cl. A61B 5/00

(52) U.S. Cl. 600/316; 600/310

(57) ABSTRACT

An optical coherence tomography based, non-invasive imaging and analysis system, includes an optical source and a compact rigid optical signal processing system which provides a probe and a reference beam. It also includes a means that applies the probe beam to the target to be analyzed, recombines the beams interferometrically and translates a rigid optical signal processing system. It further includes electronic control and processing systems.

### CROSS REFERENCES TO RELATED APPLICATIONS

This application, docket number JH040924, claims priority from provisional application, Ser. No. 60/505,464 entitled “A Compact Non-invasive Analysis Syasem” filed on Sep. 24, 2003.

This application relates to provisional application Ser. No. 60/602,913 filed on Aug. 19, 2004 titled “A Multiple Reference Non-invasive Analysis System”, the contents of which are incorporated by reference as if fully set forth herein. This application also relates to utility patent application Ser. No. 10/870,121 filed on Jun. 17, 2004 titled “A Non-invasive Analysis System”, the contents of which are incorporated by reference as if fully set forth herein. This application also relates to utility patent Ser. No. 10/870,120 filed on Jun. 17, 2004 titled “A Real Time Imaging and Analysis System”, the contents of which are incorporated by reference as if fully set forth herein.

### FIELD OF THE INVENTION

The invention relates to non-invasive optical analysis and imaging. It also relates to quantitative analysis of concentrations specific components in a target. Such components include analytes, such as glucose.

### BACKGROUND OF THE INVENTION

Non-invasive analysis is a valuable technique for acquiring information about systems or targets without undesirable side effects, such as damaging the system being analyzed. In the case of analyzing living entities, such as human tissue, undesirable side effects of invasive analysis include the risk of infection along with pain and discomfort associated with the invasive process.

In the particular case of measurement of blood glucose levels in diabetic patients, it is highly desirable to measure the blood glucose level frequently and accurately to provide appropriate treatment of the diabetic condition as absence of appropriate treatment can lead to potentially fatal health issues, including kidney failure, heart disease or stroke.

A non-invasive method would avoid the pain and risk of infection and provide an opportunity for frequent or continuous measurement. Non-invasive analysis based on several techniques have been proposed. These techniques include: near infrared spectroscopy using both transmission and reflectance; spatially resolved diffuse reflectance; frequency domain reflectance; fluorescence spectroscopy; polarimetry and Raman spectroscopy.

These techniques are vulnerable to inaccuracies due to issues such as, environmental changes, presence of varying amounts of interfering contamination, skin heterogeneity and variation of location of analysis. These techniques also require considerable processing to de-convolute the required measurement, typically using multi-variate analysis and have typically produced insufficient accuracy and reliability.

More recently optical coherence tomography (OCT), using a Super-luminescence diode (SLD) as the optical source, has been proposed in Proceedings of SPIE, Vol. 4263, pages 83-90 (2001). The SLD output beam has a broad bandwidth and short coherence length. OCT is a non-invasive imaging and analysis technique. The technique involves splitting the output beam into a probe and reference beam. The probe beam is applied to the system to be analyzed (the target). Light scattered back from the target is combined with the reference beam to form the measurement signal.

Because of the short coherence length only light that is scattered from a depth within the target such that the total optical path lengths of the probe and reference are equal combine interferometrically. Thus the interferometric signal provides a measurement of the scattering value at a particular depth within the target. By varying the length of the reference path length, a measurement of the scattering values at various depths can be measured and thus the scattering value as a function of depth can be measured.

The correlation between blood glucose concentration and scattering has been reported in Optics Letters, Vol. 19, No. 24, Dec. 15, 1994 pages 2062-2064. The change of the scattering value as a function of depth correlates with the glucose concentration and therefore measuring the change of the scattering value with depth provides a measurement of the glucose concentration. Determining the glucose concentration from a change, rather than an absolute value provides insensitivity to environmental conditions.

In conventional OCT imaging or analysis systems depth scanning is achieved by modifying the relative optical path length of the reference path and the probe path. The relative path length is modified by such techniques as electro-mechanical based technologies, such as galvanometers or moving coils actuators, rapid scanning optical delay lines and rotating polygons.

All of these techniques involve moving parts, which present significant alignment and associated signal to noise ratio related problems. Non-moving part solutions include acousto-optic scanning, which, however is costly, bulky and have significant thermal control and associated thermal signal to noise ratio related problems.

Optical fiber based OCT systems also use piezo electric fiber stretchers. These, however, have polarization rotation related signal to noise ratio problems and also are physically bulky, are expensive and require relatively high voltage control systems. These aspects cause conventional OCT systems to have significant undesirable signal to noise characteristics and present problems in practical implementations with sufficient accuracy, compactness and robustness for commercially viable and clinically accurate devices.

Therefore there is an unmet need for commercially viable, compact, robust, non-invasive device with sufficient accuracy, precision and repeatability to analyze or image targets or to measure analyte concentrations, and in particular to measure glucose concentration in human tissue.

### SUMMARY OF THE INVENTION

The invention is a method, apparatus and system for a non-invasive imaging and analysis system. The invention includes an optical source and a compact rigid optical signal processing system, which provides a probe and a reference beam. It also includes a means that applies the probe beam to the target to be analyzed, recombines the beams interferometrically and translates the rigid optical signal processing system. It further includes electronic control and processing systems.

### BRIEF DESCRIPTION OF THE DRAWINGS

FIG. 1 is an illustration of the non-invasive analysis system according to the invention.

FIG. 2 is a horizontal view of the non-invasive analysis system.

FIG. 3 is a further horizontal view of the non-invasive analysis system.

FIG. 4 is a vertical illustration of the scanning system

FIG. 5 is an illustration of an alternative embodiment of the invention.

FIG. 6 is an illustration of another alternative embodiment of the invention.

FIG. 7 is an illustration of another alternative embodiment of the invention.

FIG. 8 is an illustration of another alternative embodiment of the invention.

FIG. 9 is an illustration of a two dimensional scanning system.

FIG. 10 is an illustration of an embodiment suitable for polarized beams.

FIG. 11 is an illustration of an embodiment with two opto-electronic detectors.

### DETAILED DESCRIPTION OF THE INVENTION

Conventional optical coherence tomography is based on splitting the output of a broadband optical source into a probe and reference beam and varying the relative optical path length of the reference arm to scan the target. This approach has problems and limitations described above. An alternative approach, which addresses these problems and limitations, is to use a compact optical processing system with a fixed relative optical path length, which constitutes a fixed path length interferometer, and to achieve scanning by translating the compact optical processing system.

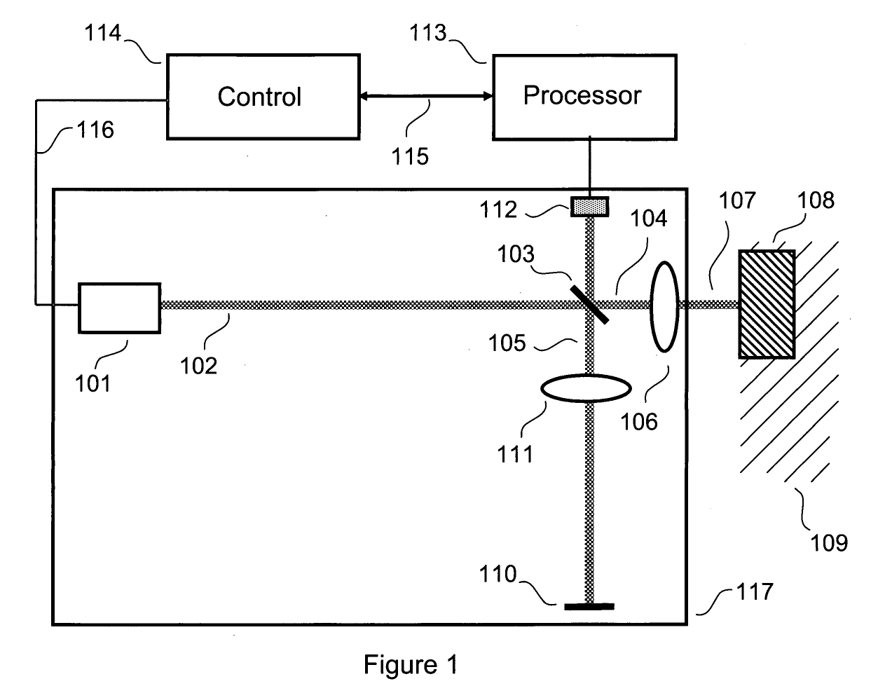

A preferred embodiment of this invention is illustrated in and described with reference to FIG. 1 where a non-invasive optical imaging and analysis system is shown. The system includes a fixed path length interferometer which provides the full operational capability of a conventional variable path length interferometer. The system includes a broadband optical source 101 such as a superluminscent diode or a mode locked laser. The optical source 101 typically includes optics to provide a collimated output beam 102, which consists of a broad band set of wavelengths.

The output beam 102, is passed through a beam splitter 103, to form a probe beam 104 and a reference beam 105. The probe beam 104 typically passes through a focusing lens 106. The focusing probe beam 107 is directed by an angled mirror 108 to focus in the target 109 below the angled mirror.

At least part of the optical signal applied to the target is scattered back and captured by the focusing lens 106. Scattering occurs because of discontinuities, such as changes of refractive index or changes in reflective properties, in the target. The captured scattered beam passes through the focusing lens 106, back to the beam splitter 103.

The reference beam 105 is also directed back to the beam splitter 103 by means of the mirror 110. The reference beam 105 also typically passes through an optional compensating focusing lens 111. The reference beam and the captured scattered beams combine interferometrically in the beam splitter 103 and the resulting signal is detected by the opto-electronic detector 112. Although typically referred to as a beam splitter the optical element 103 operates as an optical combining element, in that it is in this element that reference beam and captured scattered beam combine interferometrically.

A meaningful interferometric signal only occurs with interaction between the reference beam and light scattered from a distance within the target such that the total optical path lengths of both reference and probe paths are equal or equal within the coherence length of the optical beam.

With the exception of the angled mirror 108, the optical processing system described above may be contained on a compact micro-bench 117, including but not limited to a silicon micro-bench. By varying the distance between the micro-bench 117 and the angled mirror 108, the distance into the target from which the meaningful interferometric signal originates is varied along a line determined by the angled mirror.

This provides a method of scanning different depths within the target using an optical processing sub-system with no moving parts, allowing a rigid assembly of components, for example on a silicon micro-bench. This method removes the signal to noise and alignment problems associated conventional methods of varying the relative optical path length of the reference path length. The optical system comprised of the micro-bench and components mounted on it constitutes an optical sub-system that is a fixed path length interferometer.

With this method the meaningful interferometric signal always originates at a constant optical distance from the focusing lens 106 and thus does not necessarily require a lens with a long focal range, enabling use of a higher numerical aperture lens and also enabling the use of a pin hole in the detection path which enables higher spatial resolution and better noise discrimination.

The preferred embodiment also includes an electronic processing module 113 which interacts with an electronic control module 114 by means of electronic signals 115. The control module 114 generates control and drive signals for the system, including signals 116 to control and drive the optical source. It also controls the motion of the micro-bench 117 with respect to the angled mirror 108.

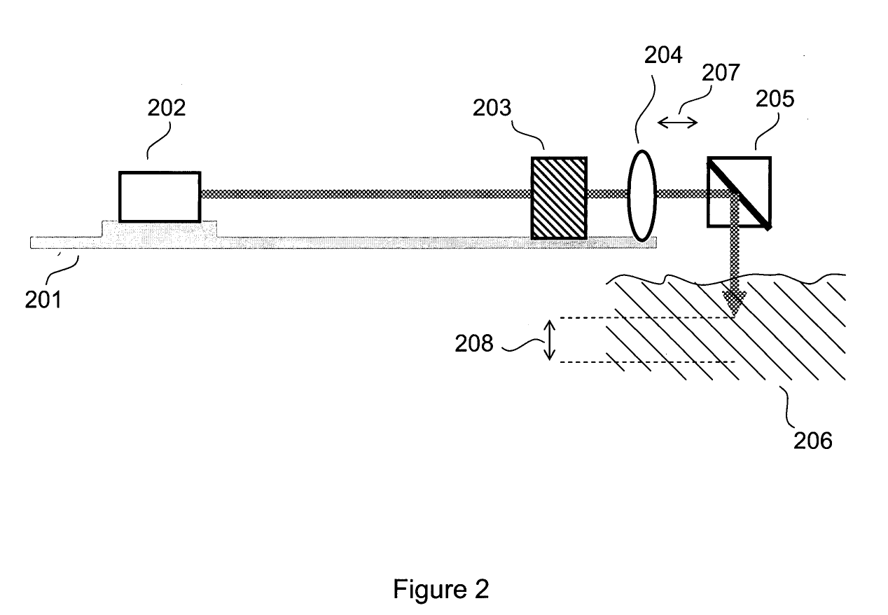

A horizontal view of the non-invasive analysis system is illustrated in FIG. 2. Shown in this horizontal view of the micro-bench 201 are the low coherence source 202, the reference mirror 203 (which obscures the beam splitter and the compensating focusing mirror) and focusing lens 204. Other components mounted on the micro-bench, but obscured in this view, include the beam-splitter which also acts as the interferometric combiner and the compensating focusing mirror. Also mounted, but obscured is the opto-electronic detector.

FIG. 2 also illustrates the angled mirror 205 which directs the optical probe signal to the target 206. The angled mirror directs the probe beam into the target in a direction perpendicular to the surface of the target. For purposes of this application, this direction shall be referred to as the vertical or longitudinal direction. The direction parallel to the surface shall be referred to as the horizontal direction.

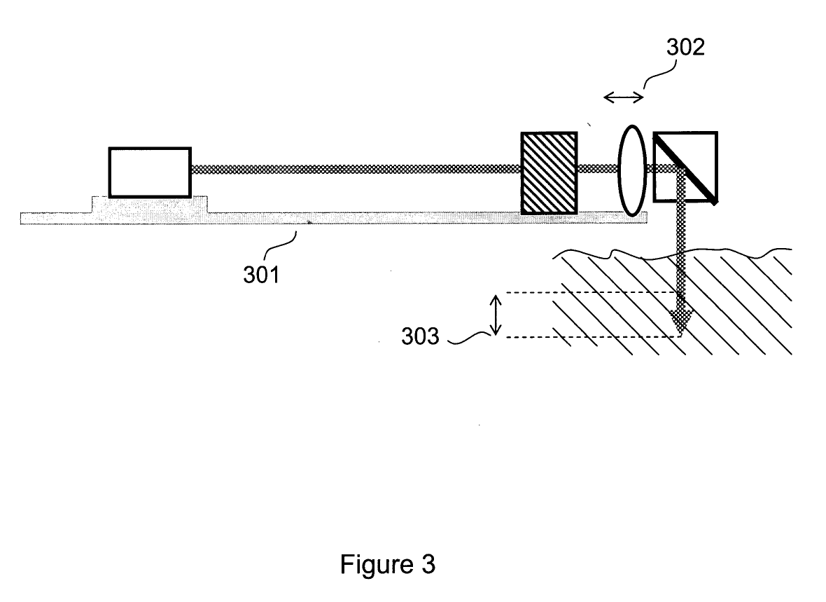

Translating the micro-bench 201 horizontally toward and away from the angled mirror 205, as indicated by 207 causes the focal point within the target to move vertically down and up in the target as indicated by 208. FIG. 2 illustrates one extreme of this motion, while FIG. 3 illustrates the other extreme of the motion. In FIG. 3 the micro-bench 301 is translated to the extreme right of the motion range 302, which causes the probe beam to be focused at the extreme lower range 303 of the analysis range within the target.

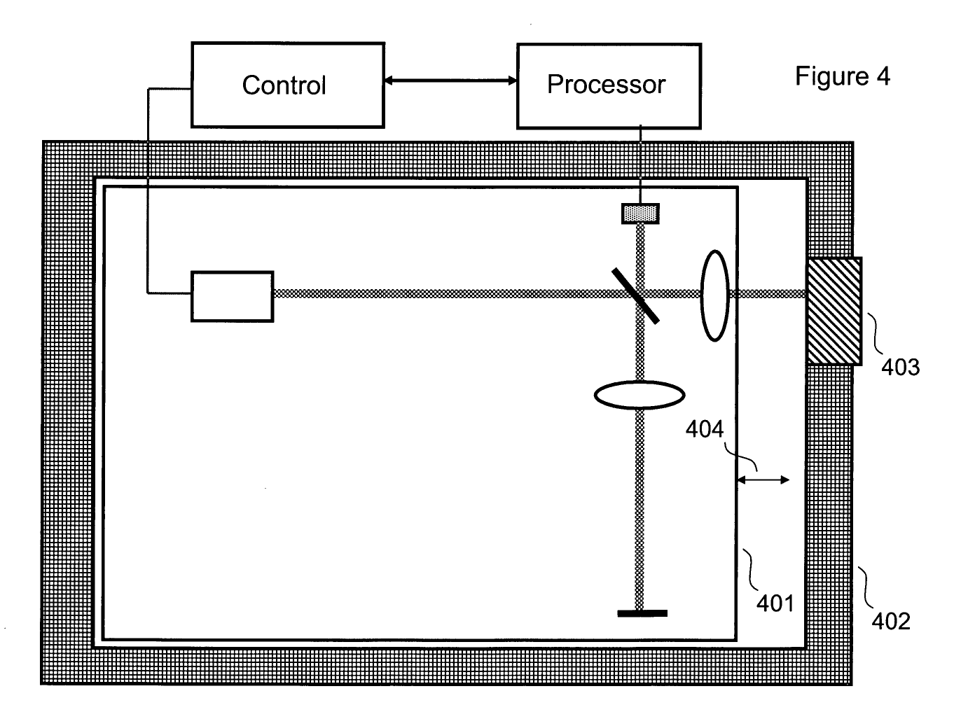

FIG. 4 illustrates an embodiment of the horizontal scanning system where the micro-bench 401 is translated within a housing 402 that contains the angled mirror 403. Translation of the micro-bench as indicated by 404 can be accomplished by conventional means such as an electro-mechanical voice coil actuator or piezo based actuator.

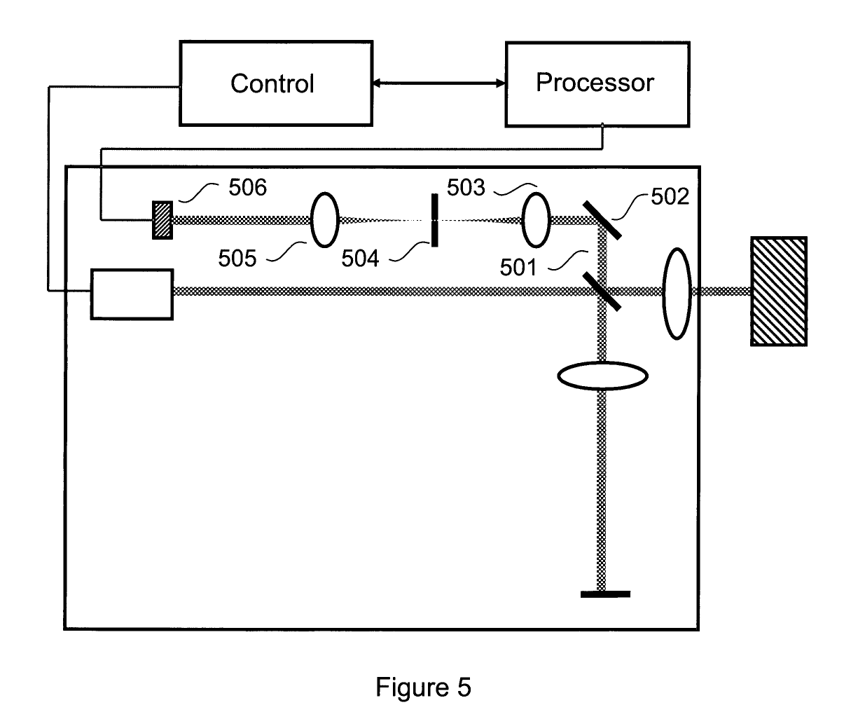

Because the desired captured returned signal always originates at the (geometric) focal point of the focusing lens, the lens does not require a long focal range as required in conventional OCT implementations. This enables using higher numerical aperture lenses. It also enables the use of a pin-hole (or pin-holes) in the detection path. This is illustrated in FIG. 5 where the interferometric optical signal 501 is redirected by a steering mirror 502 to a focusing lens 503 which focuses the signal at a pin-hole aperture 504. The output of the pin-hole is re-collimated or re-focused by lens 505 and detected by the opto-electronic detector 506.

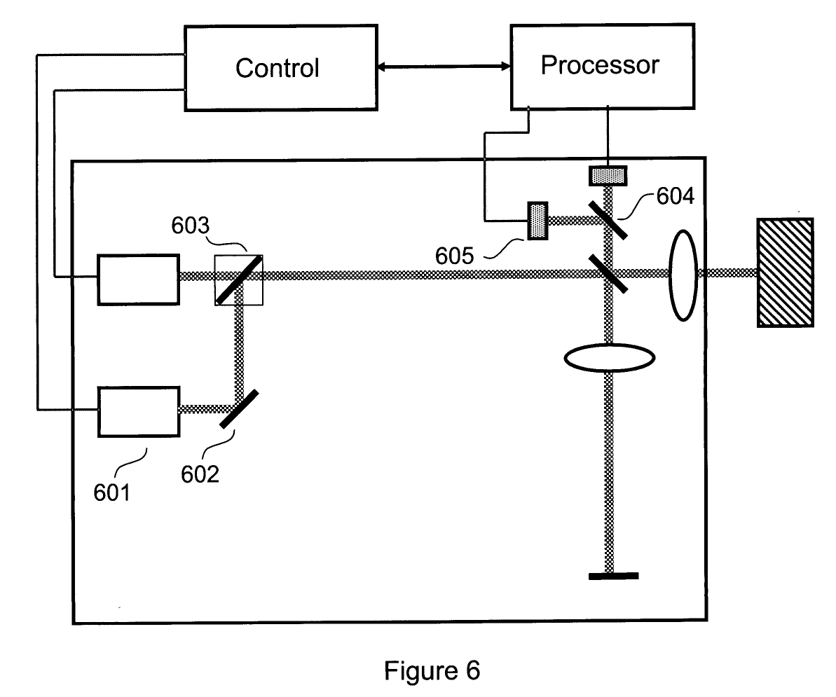

An alternate embodiment is illustrated in FIG. 6 where a second optical source 601 is shown, typically this would be at a different wavelength range than the first optical source. The output of this second optical source is directed to the output of the first optical source by means of a steering mirror 602. It is then combined with the first optical output by means of a wavelength selective mirror (or a beam splitter, used as a combiner) 603.

The interferometric signal originating from this second source is similarly separated be a second wavelength selective mirror 604 to a second detector 605. This second wavelength selective mirror can direct all or a partial amount of the second wavelength range to the second detector. Partial reflection enables higher resolution by means of the first detector. Full wavelength selection can be still achieved by selectively powering the optical sources.

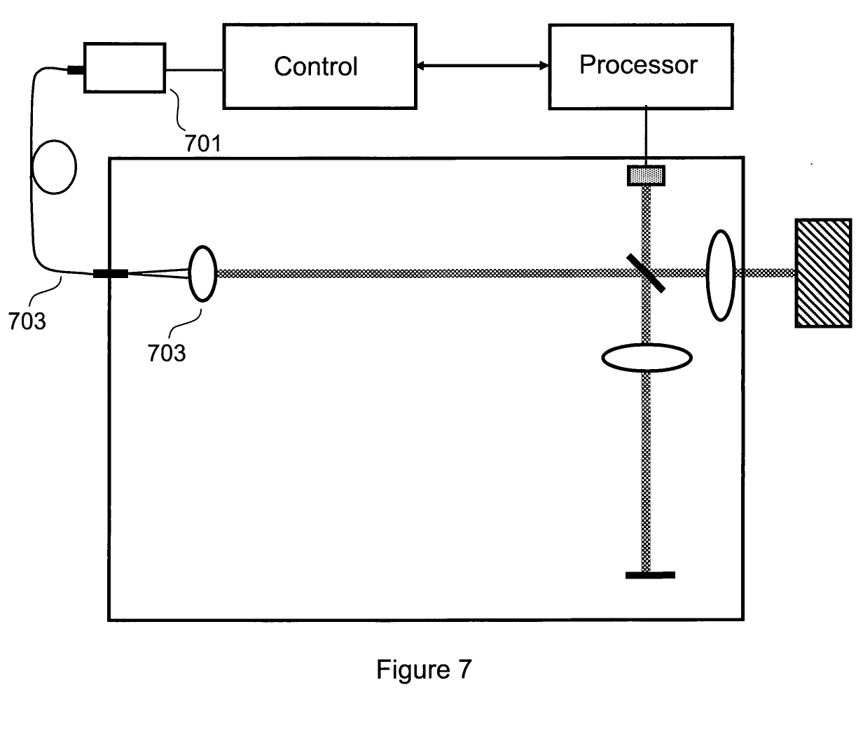

Another embodiment is illustrated in FIG. 7 where the optical source 701 is coupled to the micro-bench by means of a fiber 702. The output of the fiber is collimated by a lens 703. In this embodiment higher resolution can readily be achieved by combining, by means of fiber couplers, the outputs of multiple optical sources with adjacent or partially overlapping wavelength ranges and coupling the combined broadband optical signal to the micro-bench by means of the fiber 702. This embodiment, where the optical source is fiber coupled to the micro-bench enables a more compact system by not having the source on the compact micro-bench.

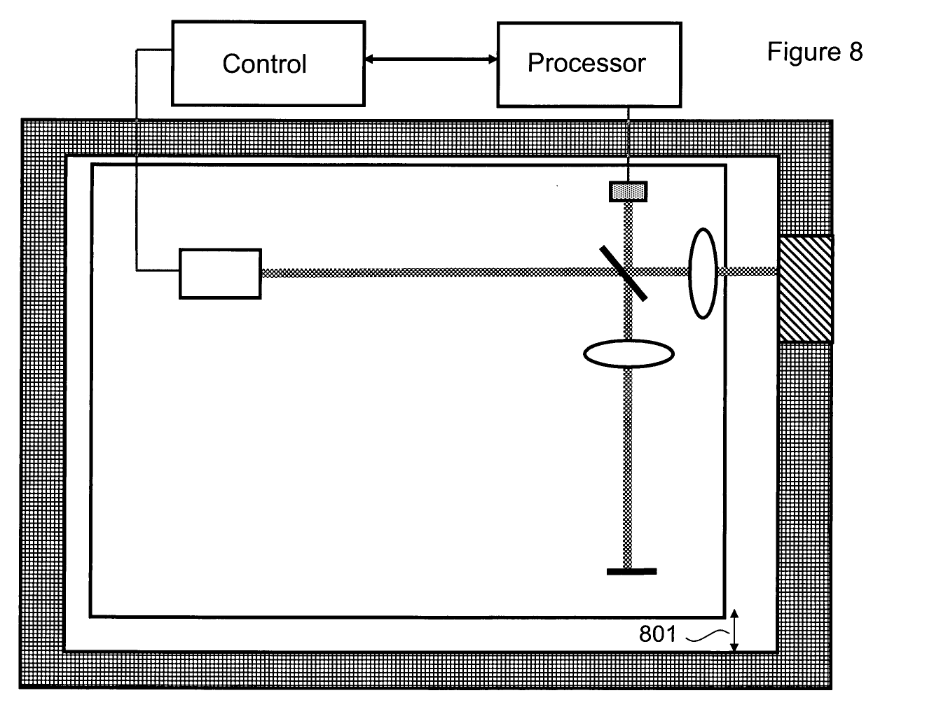

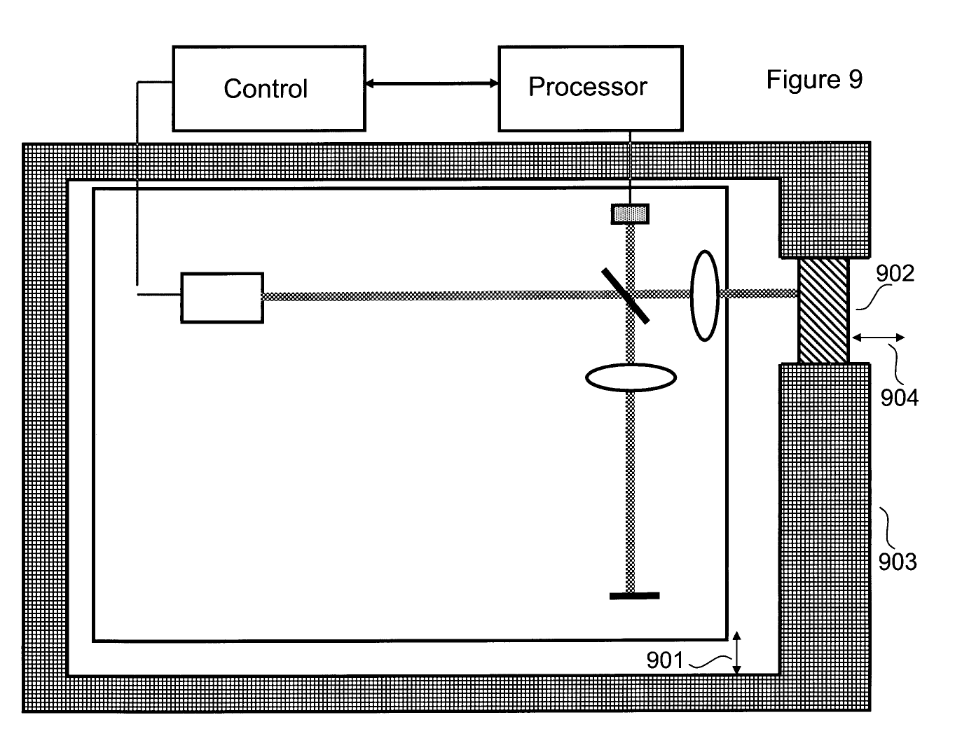

In addition to scanning in the vertical direction within the target, one dimensional scanning in the horizontal plane (parallel to the surface) can be accomplished as illustrated in FIG. 8 and indicated by 801. Two dimensional horizontal scanning can also be accomplished as illustrated in FIG. 9 where again one dimension is accomplished as indicated by 901. The second horizontal dimension scanning is accomplished by translating the angled mirror 902 with respect to the housing 903 as indicated by 904.

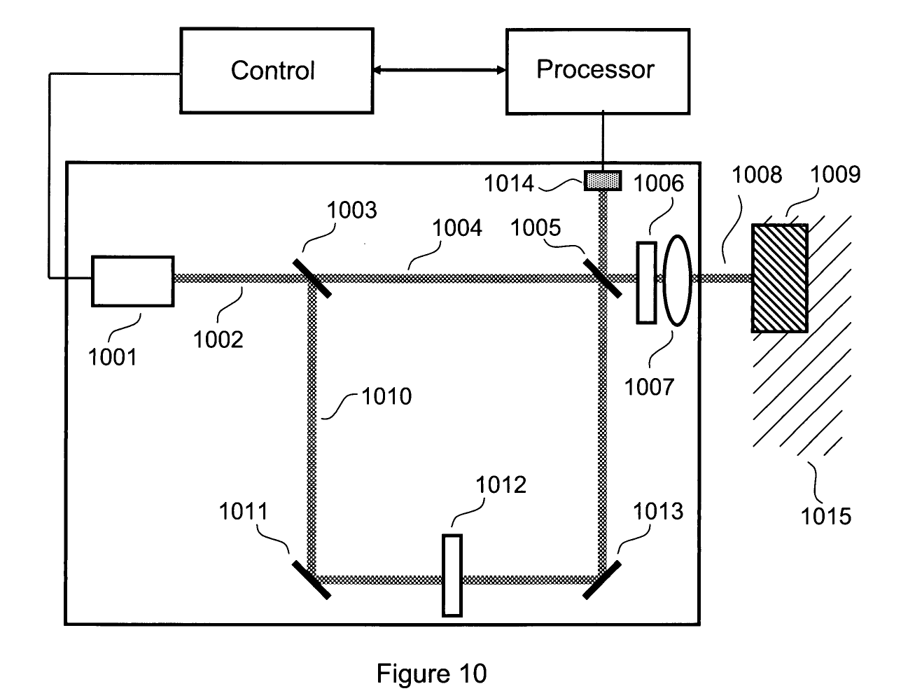

Many different configurations of the fixed path length design are possible. For example, an alternative design (suitable when using an optical source which outputs a polarized beam) is illustrated in FIG. 10. The system includes a broadband optical source 1001 such as a superluminscent diode or a mode locked laser, whose collimated and polarized output 1002, consists of a broad band set of wavelengths.

The output beam 1002, is passed through a beam splitter 1003, to form a probe beam 1004 and a reference beam 1010. The probe beam 1004 passes through a second beam splitter 1005, (such as a polarization beam splitter), through a quarter wave plate 1006 to a focusing lens 1007. The focusing probe beam 1008 is directed by an angled mirror 1009 to focus in the target 1015 below the angled mirror.

At least part of the optical signal applied to the target is scattered back and captured by the lens 1007. Scattering occurs because of discontinuities, such as changes of refractive index or changes in reflective properties, in the target. The captured scattered beam passes through the quarter wave plate 1006, back to the beam splitter 1005.

The reference beam 1010 is also directed to the beam splitter 1005 by means of steering mirrors 1011 and 1013. It also passes through a half-wave plate 1012 to rotate its plane of polarization. The reference beam and the captured scattered beams combine interferometrically in the beam splitter 1005 and the resulting signal is detected by the opto-electronic detector 1014. Although typically referred to as a beam splitter the optical element 1005 operates as an optical combining element, in that it is in this element that reference beam and captured scattered beam combine interferometrically.

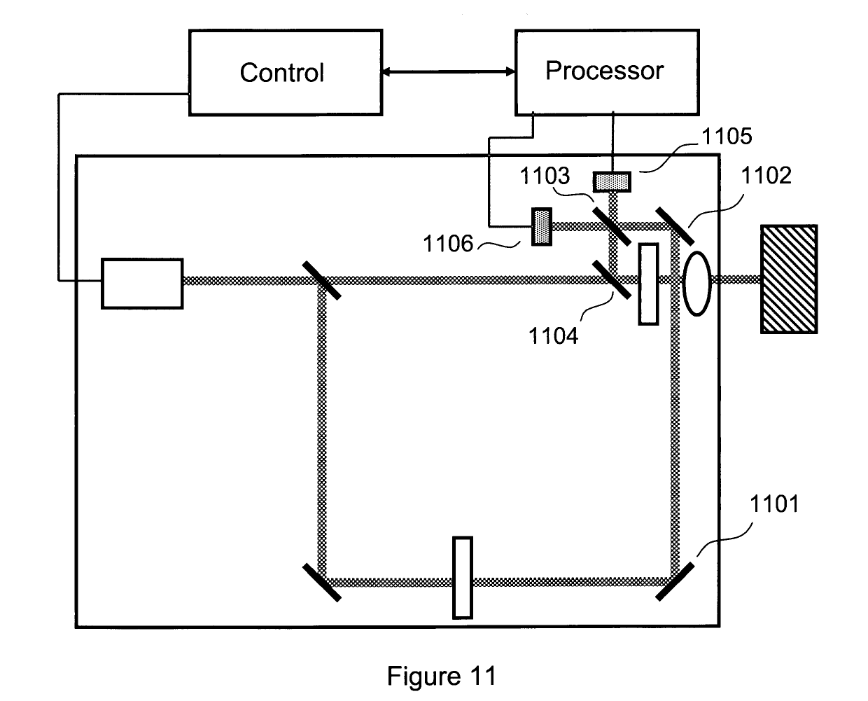

Yet another embodiment is illustrated in FIG. 11 where a balanced detection scheme using two opto-electronic detectors is shown. In this embodiment the turning mirror 1101 is re-positioned to direct the reference beam to an additional turning mirror 1102 which directs the reference beam to an additional beam splitter 1103. Substantially all of the captured returned scattered signal is directed by the polarization beam splitter 1104 to the additional beam splitter (combiner) 1103. This allows detecting complimentary interference signals by the detectors 1105 and 1106. This enables differential detection with associated noise suppression advantages.

It is understood that the above description is intended to be illustrative and not restrictive. Many of the features have functional equivalents and many variations and combinations of the above embodiments are possible and are intended to be included in the invention as being taught.

For example, using two or more optical sources can be combined with balanced detection, with either all wavelength ranges being detected simultaneously for high resolution or selectively powering (electrically turning on) individual optical sources for spectral resolution. The design of the first embodiment could be modified to include differential detection.

Other examples will be apparent to persons skilled in the art. The scope of this invention should therefore not be determined with reference to the above description, but instead should be determined with reference to the appended claims and drawings, along with the full scope of equivalents to which such claims and drawings are entitled.

What is claimed is:

1. A method for non-invasive depth analysis of a target comprising:
an optical processing sub-system consisting of a fixed path length interferometer;
focusing the optical output of said optical processing sub-system at a point within the target to be analyzed;
capturing at least part of said optical signal scattered within the target;
applying the captured scattered optical signal to said fixed path length interferometer;
detecting the interferometric output of said fixed path length interferometer;
varying the spatial relationship of said fixed path length interferometer and the target;
analyzing the detected interferometric output of said fixed path length interferometer at multiple spatial relationships of said fixed path length interferometer and the target; and
generating a non-invasive depth analysis of the target.

2. The method of claim 1, wherein the fixed path length interferometer includes a means of splitting an optical beam into at least two optical components, one of which is a reference signal, routing the two components through different fixed optical path lengths and directing both optical components to an optical combining element.

3. The method of claim 1, wherein the fixed path length interferometer includes at least one low coherence optical source.

4. The method of claim 1, wherein the fixed path length interferometer includes a fiber coupled to an external low coherence optical source.

5. The method of claims 3 and 4, wherein the low coherence optical source is a mode locked laser source.

6. The method of claims 3 and 4, wherein the low coherence optical source is a superluminscent diode.

7. The method of claim 2, wherein the fixed path length interferometer includes a means of rotating the polarization of the optical component that is a reference beam and rotating the polarization of the probe output and returned scattered optical signal.

8. The method of claim 1, wherein the optical output of said optical sub-system is focused at a point within the target to be analyzed by means of a focusing lens.

9. The method of claim 8, wherein the optical output of said optical sub-system is directed vertically along a line substantially perpendicular to the surface of the target by means of an angled mirror.

10. The method of claim 1, wherein the at least part of optical output of said optical sub-system that is scattered by discontinuities in the target.

11. The method of claim 10, wherein the discontinuities in the target are due to changes of refractive index.

12. The method of claim 10, wherein the discontinuities in the target are due to changes of reflectivities within the target.

13. The method of claim 1, wherein the scattered signal is captured by the focusing lens and returned to the fixed path length interferometer.

14. The method of claim 1, wherein the captured scattered signal is separable from the optical output of said optical sub-system by means of a polarization separator.

15. The method of claim 1, wherein the captured scattered signal is combined with a reference signal of the fixed path length interferometer.

16. The method of claim 1, wherein the captured scattered signal and the reference signal are combined interferometrically.

17. The method of claim 1, wherein the interference signal between the scattered and reference signals is detected by means of an opto-electronic detector.

18. The method of claim 1, wherein the interference signal between the scattered and reference signals is detected differentially by means of two opto-electronic detectors.

19. The method of claims 17 and 18, wherein an interference signal is focused in a pin-hole prior to detection.

20. The method of claim 1, wherein the spatial relationship between the fixed path length interferometer and the target is varied by physically moving the fixed path length interferometer.

21. The method of claim 20, wherein the spatial relationship between the fixed path length interferometer and the target is varied by varying the spatial relationship between the fixed path length interferometer and an angled mirror.

22. The method of claim 1, wherein the interference signals are detected by means of at least one opto-electronic detector at multiple spatial relationships between the fixed path length interferometer and the target.

23. The method of claim 1, wherein the detected signals are combined with electronic signals aligned with the physical motion of the fixed path length interferometer.

24. The method of claim 1, wherein the detected signals are analyzed by means of combining information from detected signals at least two temporal relationships between the captured scattered and reference signals.

25. The method of claim 24, wherein the detected signals are analyzed to determine the detected signals as a function of the depth within the target.

26. The method of claim 25, wherein the detected signals are analyzed by an electronic processing system to determine the concentration of a particular constituent or component of the target to be analyzed.

27. The method of claim 1, wherein an electronic control system coordinates the electronic signals aligned with the physical motion of the fixed path length interferometer, the detected signals and the processing system to generate a non-invasive depth analysis of the target.

28. The method of claim 1, wherein the depth analysis determines the concentration of an analyte.

29. The method of claim 28, wherein the analyte is glucose.

30. The method of claim 1, wherein the target is human tissue.

31. The method of claim 1, wherein the depth analysis provides an image of the target.

32. A system for non-invasive depth analysis of a target comprising:
an optical processing sub-system consisting of a fixed path length interferometer;
focusing the optical output of said optical processing sub-system at a point within the target to be analyzed;
capturing at least part of said optical signal scattered within the target;
applying the captured scattered optical signal to said fixed path length interferometer;
detecting the interferometric output of said fixed path length interferometer;
varying the spatial relationship of said fixed path length interferometer and the target;
analyzing the detected interferometric output of said fixed path length interferometer at multiple spatial relationships of said fixed path length interferometer and the target; and
generating a non-invasive depth analysis of the target.

33. An apparatus for non-invasive depth analysis of a target comprising:
an optical processing sub-system consisting of a fixed path length interferometer;
means for focusing the optical output of said optical processing sub-system at a point within the target to be analyzed;
means for capturing at least part of said optical signal scattered within the target;
means for applying the captured scattered optical signal to said fixed path length interferometer;
means for detecting the interferometric output of said fixed path length interferometer;
means for varying the spatial relationship of said fixed path length interferometer and the target;
means for analyzing the detected interferometric output of said fixed path length interferometer at multiple spatial relationships of said fixed path length interferometer and the target; and generating a non-invasive depth analysis of the target.

34. The apparatus of claim 33, wherein the fixed path length interferometer includes a means of splitting an optical beam into at least two optical components, one of which is a reference signal, routing the two components through different fixed optical path lengths and directing both optical components to an optical combining element.

35. The apparatus of claim 33, wherein the fixed path length interferometer includes at least one low coherence optical source.

36. The apparatus of claim 33, wherein the fixed path length interferometer includes a fiber coupled to an external low coherence optical source.

37. apparatus of claims 35 and 36, wherein the low coherence optical source is a mode locked laser source.

38. The apparatus of claims 35 and 36, wherein the low coherence optical source is a superluminscent diode.

39. The apparatus of claim 34, wherein the fixed path length interferometer includes a means of rotating the polarization of the optical component that is a reference beam and rotating the polarization of the probe output and returned scattered optical signal.

40. The apparatus of claim 33, wherein the optical output of said optical sub-system is focused at a point within the target to be analyzed by means of a focusing lens.

41. The apparatus of claim 40, wherein the optical output of said optical sub-system is directed vertically along a line substantially perpendicular to the surface of the target by means of an angled mirror.

42. The apparatus of claim 33, wherein the at least part of optical output of said optical sub-system that is scattered by discontinuities in the target.

43. The apparatus of claim 42, wherein the discontinuities in the target are due to changes of refractive index.

44. The apparatus of claim 42, wherein the discontinuities in the target are due to changes of reflectivities within the target.

45. The apparatus of claim 33, wherein the scattered signal is captured by the focusing lens and returned to the fixed path length interferometer.

46. The apparatus of claim 33, wherein the captured scattered signal is separable from the optical output of said optical sub-system by means of a polarization separator.

47. The apparatus of claim 33, wherein the captured scattered signal is combined with a reference signal of the fixed path length interferometer.

48. The apparatus of claim 33, wherein the captured scattered signal and the reference signal are combined interferometrically.

49. The apparatus of claim 33, wherein the interference signal between the scattered and reference signals is detected by means of an opto-electronic detector.

50. The apparatus of claim 33, wherein the interference signal between the scattered and reference signals is detected differentially by means of two opto-electronic detectors.

51. The apparatus of claims 49 and 50, wherein an interference signal is focused in a pin-hole prior to detection.

52. The apparatus of claim 33, wherein the spatial relationship between the fixed path length interferometer and the target is varied by physically moving the fixed path length interferometer.

53. The apparatus of claim 33, wherein the spatial relationship between the fixed path length interferometer and the target is varied by varying the spatial relationship between the fixed path length interferometer and an angled mirror.

54. The apparatus of claim 33, wherein the interference signals are detected by means of at least one opto-electronic detector at multiple spatial relationships between the fixed path length interferometer and the target.

55. The apparatus of claim 33, wherein the detected signals are combined with electronic signals aligned with the physical motion of the fixed path length interferometer.

56. The apparatus of claim 33, wherein the detected signals are analyzed by means of combining information from detected signals at least two temporal relationships between the captured scattered and reference signals.

57. The apparatus of claim 33, wherein the detected signals are analyzed to determine the detected signals as a function of the depth within the target.

58. The apparatus of claim 33, wherein the detected signals are analyzed by an electronic processing system to determine the concentration of a particular constituent or component of the target to be analyzed.

59. The apparatus of claim 33, wherein an electronic control system coordinates the electronic signals aligned with the physical motion of the fixed path length interferometer, the detected signals and the processing system to generate a non-invasive depth analysis of the target.

60. The apparatus of claim 33, wherein the depth analysis determines the concentration of an analyte.

61. The apparatus of claim 60, wherein the analyte is glucose.

62. The apparatus of claim 33, wherein the target is human tissue.

63. The apparatus of claim 33, wherein the depth analysis provides an image of the target.
